(19) United States
(12) Patent Application Publication
(10) Pub. No.: US 2025/0166854 A1
(43) Pub. Date: May 22, 2025

(54) AUTOMATED PATIENT AUTHENTICATION IN A HEALTH INFORMATION SYSTEM USING PATIENT IDENTIFICATION INSTRUMENT

(71) Applicant: MEDIREX SYSTEMS INC., Toronto (CA)

(72) Inventor: Stephen NOWAK, Niagara Falls (CA)

(21) Appl. No.: 18/841,157

(22) PCT Filed: Feb. 7, 2023
(86) PCT No.: PCT/CA2023/050154

§ 371 Date: Aug. 23, 2024

(51) Int. Cl. G16H 80/00; G06F 21/44; G16H 10/65; G16H 40/67

(52) U.S. Cl. CPC G16H 80/00; G06F 21/44; G16H 10/65; G16H 40/67

(57) ABSTRACT

A patient engagement system comprises a database and various modules for communicating with health information systems and user mobile devices. An interface module of the system communicates with a health information system to receive patient information, such as patient registration information and patient medical information, therefrom. A user application module communicates with a user mobile device to receive user information, such as patient authentication information, therefrom. The database can store the patient medical information and user profiles of authenticated patients of the patient engagement system.

### TECHNICAL FIELD

The present disclosure relates generally to patient engagement in healthcare, and more specifically to systems and methods for communicating health information between patients and other participants in patient engagement.

### BACKGROUND

Patient engagement is important for improving the quality of care received by patients and for improving an overall healthcare system. Studies have shown that patients who are actively involved with their health providers are safer, more satisfied, and less likely to experience complications that could require emergency department readmission.

Meaningful patient engagement requires forging strong relationships between patients, their family members, other informal caregivers, health professionals, and the organizations or institutions that the patients work with. To forge strong relationships, patients need to feel empowered to express their concerns and all the participants in patient engagement should be knowledgeable about each other's perspectives and experiences, the facts related to the patient's medical condition, and/or how a patient's condition may improve or get better in the future. Meaningful patient engagement also requires transparency and responsiveness, especially from the side of health professionals and the institutions that they work for. For example, health professionals need to be able to convey their apprehensions or resource limitations honestly and directly to a patient. As another example, health institutions need to be able to deliver test results directly to patients in a timely manner.

One way of improving patient engagement is to provide a patient portal (i.e. a secure online website) that provides patients with access to their own personal health information. Some currently existing web-based patient portals allow patients to view health information such as discharge summaries, immunization history, lab results, and medications. Other currently existing web-based patient portals facilitate patient and health provider interactions by allowing patients to message their doctor, schedule appointments, make payments, and request prescription refills. Despite their prevalence, currently existing patient portals share several common drawbacks.

One drawback with current patient portals is that they typically require the patient to create a secure username and password to access the patient portal. Such account creation processes are non-user friendly and can be especially challenging to patients who are not proficient with technology. Another issue with current patient portals is that they are always specific to certain health institutions or certain health service providers (i.e. each health institution or health service provider has its own patient portal). Patients typically find it tedious to make multiple accounts to gain access to multiple patient portals, so patients typically underuse or do not use existing patient portals at all. Other types of current patient portals may lack of real-time notifications, may be difficult to navigate, may not inform the patient of available hospital or health care provider resources, and may not be informative or accessible to supporters or family members who can help encourage patient engagement.

There remains a need for systems and methods that help facilitate effective communication of health information between patients and the other participants of patient engagement (e.g. health professionals, caregivers, family members, health institutions, etc.). Such systems and methods should, illustratively, be easy to use for patients and/or their guardians, compatible with the digital resources already in use by healthcare facilities, and compliant with health information privacy requirements set out by government regulators. There remains a need for improved patient engagement systems that can be operated in real time. There also remains a need for improved patient engagement systems that do not add additional overhead costs to a health facility.

The foregoing examples of the related art and limitations related thereto are intended to be illustrative and not exclusive. Other limitations of the related art will become apparent to those of skill in the art upon a reading of the specification and a study of the drawings.

### SUMMARY OF THE DISCLOSURE

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention relates to a patient engagement system. The system comprises one or more of an interface module, a user application module, a user authentication, a database, and a processing module. The interface module receives or may be configured to receive patient information, such as patient registration information and patient medical information, from a health information system (e.g., a hospital information system). The user application module receives or may be configured to receive user information, such as patient authentication information, and other user inputs from a mobile device. The user authentication module compares or may be configured to compare the patient registration information with the patient authentication information. The user authentication module may create or be configured to create a user profile in situations where the patient registration information matches the patient authentication information. The database stores or may be configured to store the patient medical information and the user profile. The processing module manages or may be configured to manage access to the patient medical information based on one or more access rules. The access rules may be defined at least in part by the user profile.

In some embodiments, the patient engagement system is connected to the health information system by way of a secured application programming interface (API) provided by a provider (e.g. hospital) of the health information system. In some embodiments, the user application module is connected to the mobile device by way of the internet. In some embodiments, the database is a cloud-based database residing behind one or more layers of security firewalls.

Another aspect of the invention relates to a computer-implemented method of authenticating a mobile device of a patient to provide the patient with access to a patient engagement system. The method may be performed by the patient engagement system. The system comprises receiving patient registration information from a health information system over a first network, receiving patient authentication information from the mobile device of the patient over a second network, and comparing the patient registration information with the patient authentication information. The patient authentication information is typically obtained from a patient identification instrument issued to the patient by a provider of the health information system. Where the patient registration information matches the patient authentication information, the mobile device is granted with access to the patient engagement system. The provider of the health information system may be a hospital, and the patient identification instrument may be a hospital wristband.

The patient authentication information may be obtained by the user device reading a visual machine-readable code printed on the patient identification instrument. In some embodiments, the patient authentication information may be decoded from the visual machine-readable code at the user device before the patient authentication information is received at the patient engagement system. In other embodiments, the patient authentication information may be decoded from the visual machine-readable code at the patient engagement system.

Where the patient registration information matches the patient authentication information, a patient profile of the patient may be created based on the patient registration information. The patient profile may include a contact information of the patient obtained from the patient registration information. In some embodiments, a confirmation message may be communicated to the user device of the patient through the contact information.

In some embodiments, the first network is a secure private network. In some embodiments, the second network is a public network. In some embodiments, the first network and the second network are both public networks (e.g., the internet). In some embodiments, the patient registration information is received from the health information system over the first network in real-time. For example, the patient registration information may be retrieved from the health information system immediately upon receipt of the patient authentication information.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive. Features and advantages of the embodiments of the present invention will become apparent from the following detailed description, taken with reference to the appended drawings in which:

FIG. 1 is a block diagram of a patient engagement system according to an example embodiment.

FIG. 1A schematically illustrates the FIG. 1 patient engagement system in operation with a hospital information system and other health information systems.

FIG. 2 illustrates an example user interface of a an end-user software application of the FIG. 1 patient engagement system.

FIGS. 2A-2F illustrate various an exemplary features provided by the application as displayed by the FIG. 2 user interface.

FIG. 3 is a flow diagram illustrating an example of a patient's assessment experience in a hospital.

### DETAILED DESCRIPTION

The description which follows, and the embodiments described therein, are provided by way of illustration of examples of particular embodiments of the principles of the present invention. These examples are provided for the purposes of explanation, and not limitation, of those principles and operation of the invention.

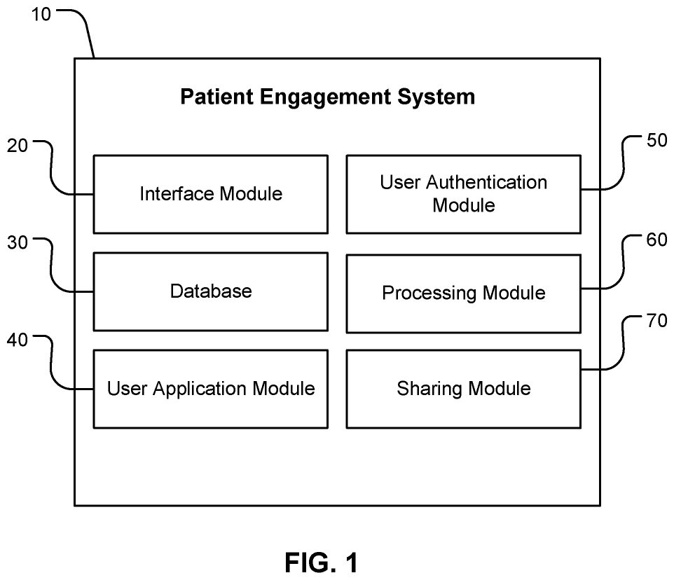

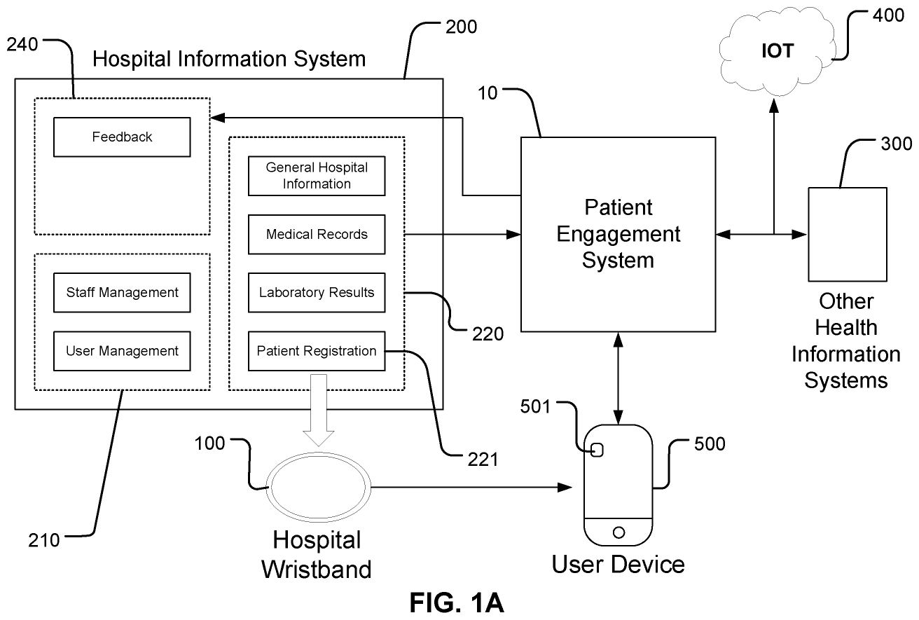

FIG. 1 is a block diagram of a patient engagement system 10 according to an example embodiment. Patient engagement system 10 may be adapted to communicate with a wide variety of different types of health information systems to provide a single point of access for patients to view their health information and/or interact with other patient engagement participants. Unless context dictates otherwise, the term “health information system” (as used herein) should be interpreted to include hospital information systems, drug information systems, any system that collects, stores, manages and/or transmit a patient's electronic medical record (EMR), a hospital's operational management system, and any system that supports healthcare policy decisions. Unless context dictates otherwise, the term “patient engagement participants” (as used herein) should be interpreted to include the patient, health professionals (e.g. doctors and staff), caregivers, family members, and health institutions or facilities.

Illustratively, patient engagement system 10 can be operated independently by a third-party service provider so it does not introduce additional overhead costs to existing health institutions or facilities. Patient engagement system 10 can encourage communication between patients and other patient engagement participants, thereby resulting in more engaged patients and/or improved health outcomes for the patient.

As depicted in FIG. 1, patient engagement system 10 comprises an interface module 20 configured to retrieve information from various health information systems. For example, interface module 20 may be connected to a hospital information system 200 through a network such as the internet or a private network as depicted in FIG. 1A. Although not necessary, interface module 20 is typically connected to hospital information system 200 by way of a secure and encrypted application programming interface (API). The API may be provided by the health information system provider (e.g., hospitals, clinics, pharmacies, etc.) and/or the third party provider of patient engagement system 10. The API may include features (e.g., security tokens, or the like) that ensure secure connection of interface module 20 to hospital information system 200 and/or features that help decrypt the encrypted data from hospital information system 200.

Optionally, interface module 20 may be connected to other health information systems 300 such as drug information systems and/or online networks 400 such as the Internet of Things to retrieve information therefrom and/or communicate information thereto. Some of the retrieved information may be stored in a database 30 of patient engagement system 10. Database 30 may be a cloud-based database. Database 30 may reside behind one or more layers of security firewalls. Access to database 30 is typically restricted to authorized users of patient engagement system 10 (e.g., users authorized by the provider of patient engagement system 10, users authorized by the provider of hospital information system 200 or other health information systems 300, users authorized by a hospital, users authorized by the government, etc.).

In some cases, a health information system stores information that can be categorized as private information (i.e. information accessible only to the institution), semi-private information (i.e. information accessible only to certain individuals such as the patient), or public information (i.e. information accessible to everyone). For example, hospital information system 200 may store private information 210 such as user management information and staff management information as well as semi-private and public information 220 such as general hospital information, patient medical records, patient laboratory results, and patient registration information. In such cases, interface module 20 may be configured to access only the semi-private or public information 220 stored on hospital information system 200, but not the private information 210. That is, interface module 20 may be configured to interface with only certain sub-systems of hospital information system 200, thereby isolating the private information 210 from the accessible information 220 stored on hospital information system 200.

In some embodiments, interface module 20 is configured to receive information from but not transmit information to a health information system. That is, interface module 20 may be operated to receive information from a health information system but cannot transmit information to the same health information system (i.e., the health information systems may be “READ-ONLY” from the perspective of patient engagement system 10). For example, interface module 20 may be operated to receive (but not modify) accessible information 220 stored in hospital information system 200 in the example illustrated in FIG. 1. This can help ensure that patient engagement system 10 is prohibited from modifying certain important information stored in the health information system, which can be essential for compliance with government regulations in some cases.

In other embodiments, interface module 20 may be operated to transmit information to a health information system, but only to limited sub-systems of the health information system. For example, interface module 20 may be configured to transmit feedback information (e.g. feedback information provided by a patient to patient engagement system 10) to a feedback sub-system 240 of a health information system. As depicted in FIG. 1, the feedback sub-system 240 may be isolated from the sub-systems that store information 210, 220, thereby improving security of the health information system. In some embodiments, the feedback information is collected from the users of patient engagement system 10 and stored in database 30 before it is transmitted to the health information system. Feedback information stored in database 30 may be queried (e.g., either automatically or manually by the provider of patient engagement system 10) such that only certain collected feedback information is transmitted to the health information system.

A user application module 40 of patient engagement system 10 is configured to communicate the information received by interface module 20 to a user device 500 (e.g. a computer, a smartphone, a smartwatch, a mobile device, etc.) of a patient and/or an individual authorized by the patient (e.g. a family member), as described in more detail below. In some embodiments, user application module 40 is configured to transmit information to user device 500 in a format that is displayable on a graphical user interface (GUI) 510 of a user application 501 of user device 500. User application 501 is typically provided by the third party provider of patient engagement system 10 as a part of patient engagement system 10, although this is not necessary. User application 501 may include browser-based applications, web-based applications, desktop applications, mobile applications, or any other suitable software applications.

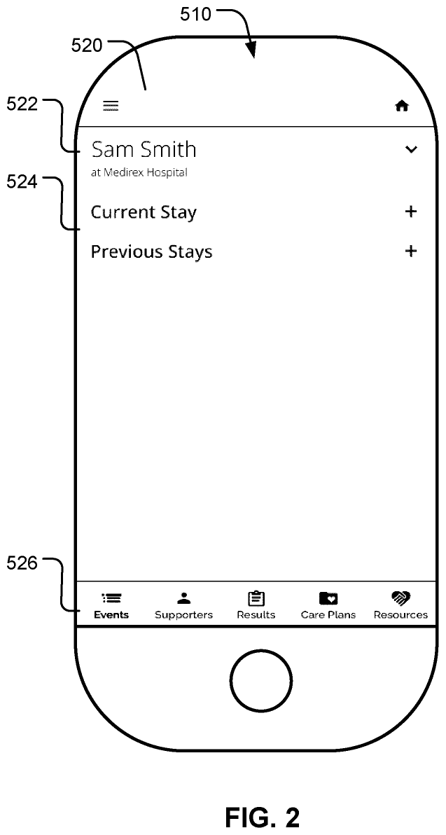

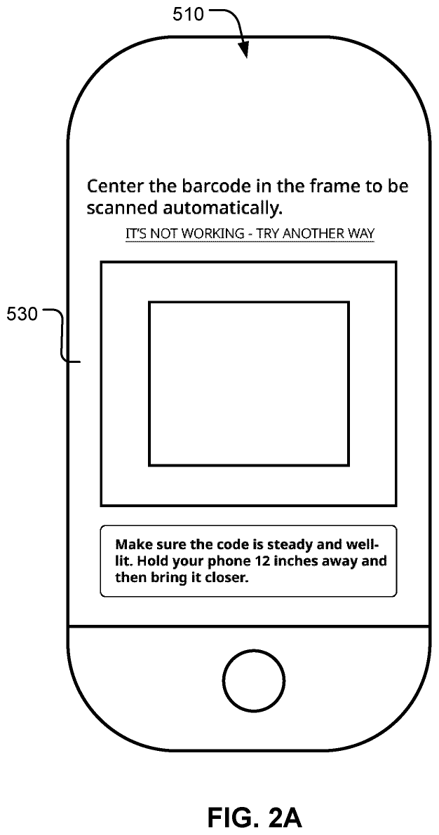

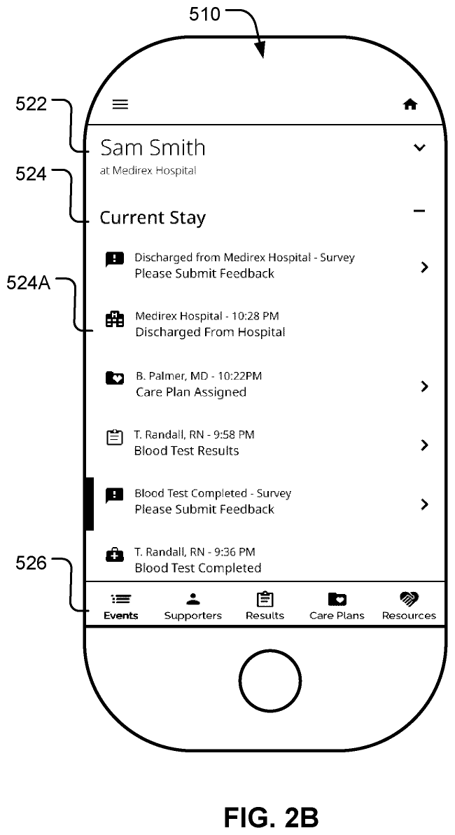

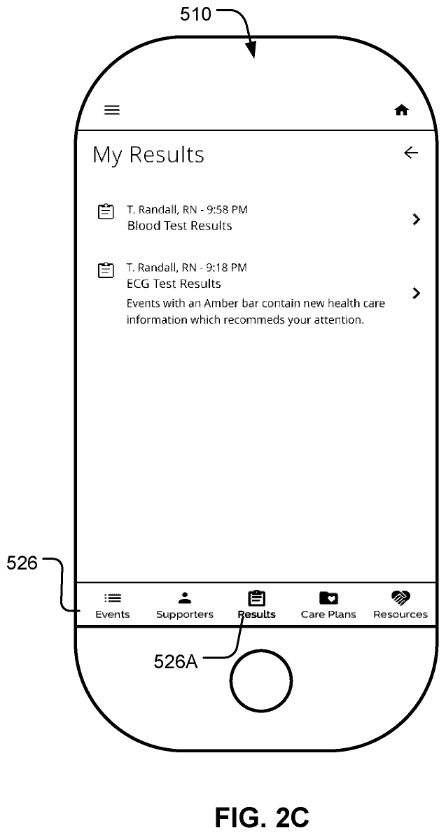

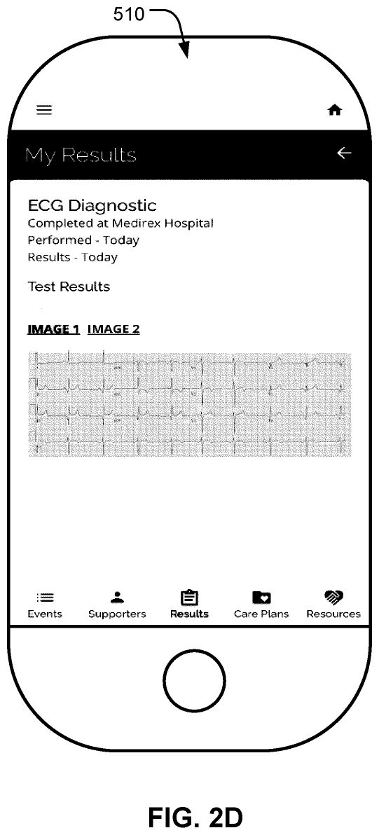

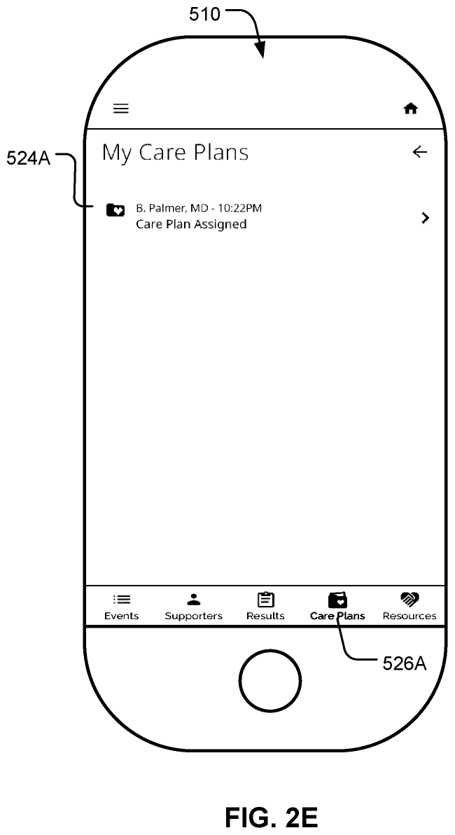

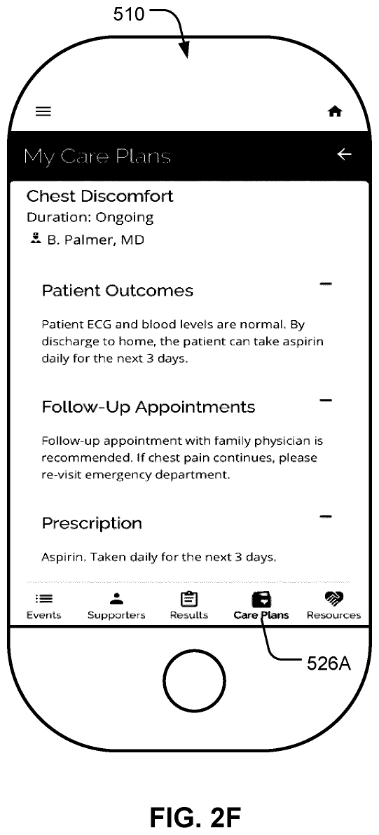

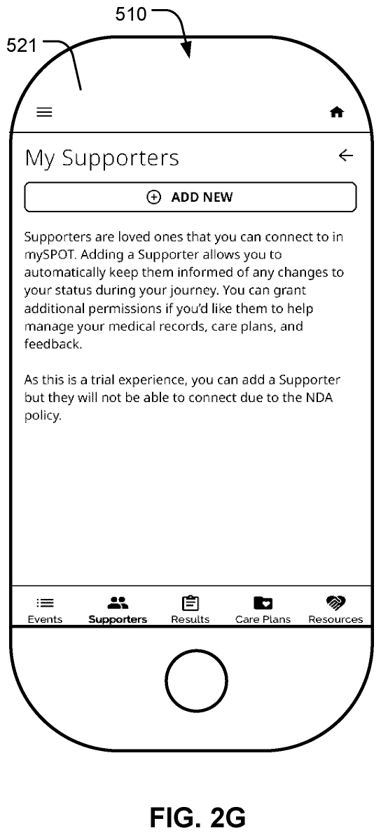

FIG. 2 illustrates a homepage 520 of an exemplary user interface 510 of mobile browser-based application 501. In the example embodiment illustrated in FIG. 2, homepage 520 comprises a patient identity field 522 for displaying the identity (e.g. name) of the patient, an events field 524 for tracking and displaying the patient's various visits to a health institution, and a menu 526 for navigating between pages corresponding to features provided by patient engagement system 10 and accessed through user application 501. Menu 526 may be displayed as a horizontal sidebar menu within user interface 510 as illustrated in FIG. 2, although this is not necessary. Menu 526 may be displayed within user interface 510 as a vertical sidebar menu, a drop down menu, a list menu, a tab menu, etc.

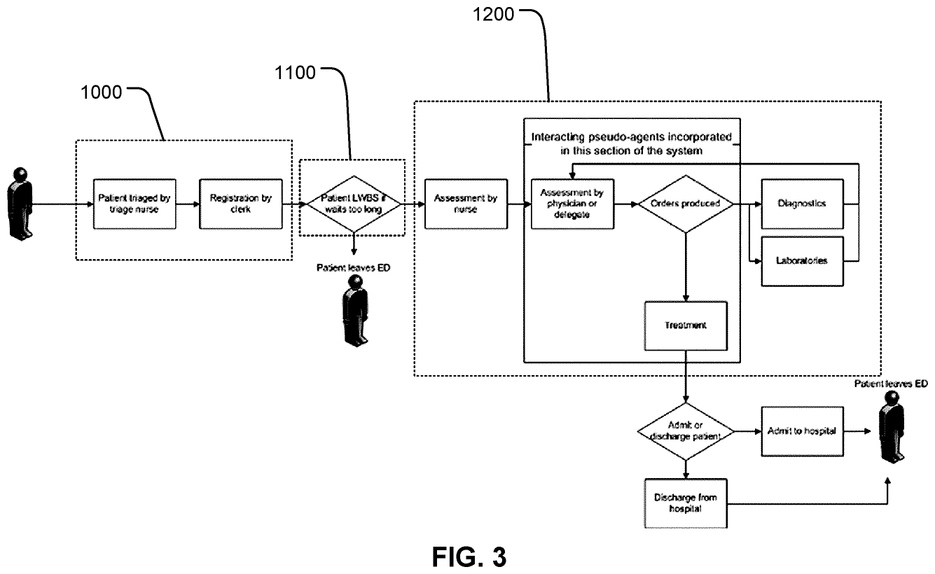

To access homepage 520 of user interface 510 and the features provided by user application 501, a user (e.g. a patient) must first be authenticated by patient engagement system 10. This may be illustrative due to the highly personal and confidential nature of health-related information. Patient engagement system 10 comprises a user authentication module 50 which facilitates the user authentication process. User authentication module 50 may be operated to authenticate a patient to provide them with a single point of access to their health information, which may be stored across various platforms (e.g. various health information systems such as hospital information system 200) that are in communication with patient engagement system 10. Illustratively, user authentication module 50 enables rapid and user-friendly patient authentication to provide a patient with easy access to their health information through patient engagement system 10. Aspects of user authentication module 50 are described in more detail below with reference to FIG. 3.

FIG. 3 is a flow diagram illustrating an example of a patient's experience at a health facility such as a hospital. At initial step 1000, the patient enters the health facility and is checked in to the facility. The step 1000 check-in process can be different for different types of health facilities and/or different situations. For example, a patient walking in to a medical clinic may be checked in by a receptionist of the medical clinic. As another example, a patient entering the emergency department of a hospital may be triaged by a triage nurse and subsequently registered in the hospital by a clerk at step 1000 as depicted in FIG. 3

In some cases, the step 1000 check-in process involves the health facility issuing a patient identification instrument 100 (e.g. see FIG. 1A) to the patient. Patient identification instrument 100 may be a number tag, a patient identification card, a wristband or bracelet, etc. Patient identification instrument 100 may be adapted by the health facility to carry important health care information such as a patient's particulars, conditions, allergies, medications (i.e. the type of medicine that should be administered, the medicine dosage, etc.), etc. For example, patient identification instrument 100 may comprise a visual machine-readable code (e.g. the code may be printed on patient identification instrument 100), such as a barcode or a QR code, that can be scanned by a scanner (e.g. a barcode scanner, a QR code scanner, the camera of a phone, a mobile device, etc.) to return the information carried by patient identification instrument 100. Such patient identification instruments 100 are commonly used in hospitals to help reduce the possibility of medical errors and/or keep track of a patient's medical condition.

After completing the step 1000 check-in process, the patient proceeds to a waiting step 1100. As depicted in FIG. 3, waiting step 1100 typically comprises a decision block where the patient either waits until they are seen by a doctor or health professional or leaves without being seen (LWBS). If the wait time to see the doctor or health professional is too long or if the patient feels neglected during waiting step 1100, then it is relatively likely that the patient will choose to leave the health facility without being seen. On the other hand if the wait time to see the doctor or health professional is reasonable or if the patient feels engaged during waiting step 1100, then it is relatively likely that the patient will choose to wait and proceed to the next assessment and treatment step 1200.

One aspect of the invention relates to a patient engagement system 10 that may be operated to increase patient engagement during waiting step 1100 and anytime thereafter. Patient engagement system 10 may be operated to authenticate a patient at their point of entry into a health facility (e.g. during initial check-in step 1000 described above), thereby providing the patient with direct access to useful health information across multiple approved platforms as the patient waits for treatment (e.g. during waiting step 1100 described above) and anytime thereafter.

To authenticate a patient at their point of entry into a health facility, interface module 20 of patient engagement system 10 is configured to receive patient registration information 221 from hospital information system 200 in real time. That is, interface module 20 may receive patient registration information 221 from hospital information system 200 immediately after such patient registration information 221 has been entered in hospital information system 200 at block 1000. In some embodiments, patient registration information 221 is automatically pushed from hospital information system 200 to interface module 20 upon the information being entered in hospital information system. In other embodiments, interface module 20 is configured to retrieve patient registration information 221 from hospital information system 20 upon a triggering event. For example, interface module 20 may send a request to hospital information system 20 to retrieve patient registration information 221 therefrom upon user application module 40 receiving patient authentication information from mobile device 500, as described in more detail elsewhere herein.

Hospital information system 200 may communicate patient registration information 221 to patient engagement system 10 through a network such as the internet or a secured network. Patient registration information 221 may include information such as patient name, patient date of birth, patient address, etc. The received patient registration information 221 may be saved in database 30 or temporarily stored in a cache of patient engagement system 10.

At the same time as or before interface module 20 receives patient registration information 221 from hospital information system 200, the patient that has just checked-in to the health facility at step 1000 may be prompted to authenticate themselves on patient engagement system 10 through their own personal mobile device 500 (e.g. a smartphone, a tablet, a smartwatch, etc.). For example, the registration clerk who is checking the patient into the health facility may instruct the patient to install and/or launch user application 501 on their mobile device 500 to access the user authentication features provided therein. As another example, the patient identification instrument 100 (e.g. hospital wristband or armband) issued to the patient at check-in step 1000 may contain instructions that prompt the patient to install and/or launch user application 501 on their mobile device 500 to access the user authentication features provided therein.

Upon accessing user application 501, the user may be prompted by user application 501 to authorize patient engagement system 10 to temporarily store and use the patient information 220 received from hospital information system 200. The authorization request may be provided to the user in a form that is compliant with applicable government data privacy and data residency regulations, such as the General Data Protection Regulation (GDRP), the Personal Information Protection and Electronic Documents Act (PIPEDA), the Health Insurance Portability and Accountability Act (HIPAA), and other like data privacy and data residency regulations in the jurisdiction where patient engagement system 10 and/or applicable health care facility reside.

FIG. 2A depicts an exemplary authenticator 530 of user application 501, as displayed on user interface 510. In the example illustrated in FIG. 2A, authenticator 530 instructs the built-in camera of mobile user device 500 to capture an image of patient identification instrument 100. Authenticator 530 may instruct the camera to capture an image when the user hovers the camera of mobile device 500 over patient identification instrument 100 and/or when authenticator 530 detects patient identification instrument 100 in the view of the camera. Authenticator 530 may be configured to instruct the camera to capture an image of only portions of patient identification instrument 100 that carry relevant health information. For example, authenticator 530 may be configured to instruct the camera to capture an image of a QR code printed on a wristband 100 issued to the patient.

After capturing an image carrying health information, authenticator 530 communicates the captured image and/or the information contained therein, including patient authentication information, to user application module 40 of patient engagement system 10 (e.g. over a wireless network such as the internet). In some embodiments, authenticator 530 decodes the patient authentication information from the captured image before communicating the decoded image to patient engagement system 10. In other embodiments, authenticator 530 transmits the captured image to user application module 40 of patient engagement system 10, and user authentication module 50 is configured to analyze (e.g. read, decode, etc.) the captured image to obtain the patient authentication information. User authentication module 50 compares the patient authentication information carried by the patient identification instrument 100 (i.e. the information transmitted by user device 500 to authentication module 50) with the patient registration information retrieved from health information system 200. If the patient authentication information carried by the patient identification instrument 100 matches the patient registration information received by interface module 20, then user authentication module 50 confirms the user's identity and grants user device 500 with access to the corresponding health information of the user contained in patient engagement system 10.

Illustratively, authenticator 530 functions in connection with user authentication module 50 to authenticate a patient's user device 500 with patient engagement system 10 without requiring the patient to manually create their own user account and/or password. This provides a simple and user-friendly way for patients to access patient engagement system 10, thereby encouraging patient engagement across all health facilities connected to patient engagement system 10. In addition, since interface module 20 of patient engagement system 10 is configured to retrieve or otherwise receive information from health information systems in real time, a patient's user device 500 can be authenticated with patient engagement system 10 almost instantaneously upon creation of the patient's profile in the health information system. This can be especially important in hospital emergency room settings, where it is desirable to provide the waiting patients with access to patient engagement system 10 immediately upon their check-in to reduce the likelihood of the patients leaving without being seen.

Illustratively, patient engagement system 10 leverages the issuance of a patient identification instrument 100 (e.g. a hospital wristband) to encourage the patient and their supporters to participate in patient engagement. Patient engagement system 10 enables a patient to create, at the point of issuance of the patient identification instrument 100, a user profile with patient engagement system 10 to document their medical journey. In some embodiments, once the profile is created with patient engagement system 10, the patient can access their profile through user application 501 at any time thereafter (i.e. even if the patient identification instrument 100 is disposed of after assessment and treatment step 1200).

In some embodiments, user authentication module 50 is configured to implement a two-factor authentication procedure to provide additional security. In such embodiments, user authentication module 50 is configured to perform an additional (i.e. second factor) authentication step after matching the patient registration information 221 received at interface module 20 (e.g., information retrieved from hospital information system 200) with the information communicated to user application module 40 (i.e. information carried on patient identification instrument 100 and obtained by user device 500). The additional authentication step may comprise verifying the matched contact information of the user of user device 500. For example, user authentication module 50 may be configured to obtain the matched contact information (e.g. cell phone number, email, etc.) of the patient and use the matched contact information to send a confirmation message to user device 500. For example, user authentication module 50 may obtain the matched cell phone number of the patient and send a voice or text message containing a verification code to the matched cell phone number. As another example, user authentication module 50 may obtain the matched email address of the patient and direct patient engagement system 10 to send an email containing a confirmation link to the matched email address.

Upon receiving the confirmation message on its user device 500, the patient may be prompted to confirm their identity by entering the confirmation message into user application 501. In some embodiments, authenticator 530 of user application 501 is configured to automatically detect receipt of the confirmation message if user application 501 is provided on the same user device 500 as the user device 500 that is receiving the confirmation message. This can help further simplify the two-factor authentication procedure. Authenticator 530 then communicates the entered or detected confirmation message back to user application module 40 of patient engagement system 10, where the communicated confirmation message is checked with the originally generated confirmation message to verify the identity and user device 500 of the patient. Once the patient is verified by the two-factor authentication procedure, the patient is granted access to their private health information through user application 500.

Referring now to FIGS. 2B-2G, various features supported by an example embodiment of patient engagement system 10, as provided through user application 501, are shown. FIG. 2B illustrates examples of the types of information that may be associated with the events field 524 of user interface 510. Events field 524 may be filled with various “events” stored in patient engagement system 10, where each event may correspond to a visit or stay with a health facility. Each event may include sub-events 524A that correspond to the activities that occurred during the event or action items related to the event. The sub-events 524A may be displayed on the homepage 520 of user interface 510 in the form of a drop down list as illustrated in FIG. 2B or in any other suitable form. Each sub-event 514A my include a brief description and/or a time stamp as illustrated in FIG. 2B. Examples of the types of sub-events 524A include, but are not limited to: the admission of the patient to the health facility, the completion of a medical test, the results of a medical test, the request of a feedback form, the assignment of a care plan, and the discharging of the patient from the health facility.

In some embodiments, patient engagement system 10 is configured to deliver a notification to an authenticated user device 500 upon retrieval or receipt of health information corresponding to a sub-event 524A. For example, user application module 40 may be configured to communicate a push notification to user application 501 and user application 501 may subsequently display the push notification on the authenticated user device 500. Since interface module 20 of patient engagement system 10 can receive information from various health information systems 200, 300 in real time, user application module 40 can communicate the received information to software application 501 in real time to provide authenticated patients with real time updates through their user device 500.

In some embodiments, patient engagement system 10 comprises a processing module 60 that may be configured to categorize some of the received health information into various categories 526A of sub-events 524A. In some embodiments, some or all of the various categories 526A are provided in menu 526 for quick access. For example, sub-events 524A like medical test results (e.g. blood test results, ECG test results, etc.) may be categorized under the “Results” category 526A, and the “Results” category 526A may be displayed in the menu 526 of user interface 510 as illustrated in FIG. 2C. This can conveniently allow an authenticated patient to view all of their medical test results on their user device 500 by navigating to the “Results” page corresponding to the “Results” category 526A displayed in menu 526.

In some embodiments, user application 501 and interface 510 are configured to support graphic renderings corresponding to important health information received from health information system 200. For example, mobile application 501 and user interface 510 may be configured to display medical images (e.g. ECG cardiograms, X-ray images, MRI images, etc.) associated with certain medical test results as illustrated in FIG. 2D.

Other example non-limiting types of categories 526A can include care plans, general events, additional resources, etc. FIGS. 2E-F illustrate an example of a sub-event 524A that can be categorized under a “Care Plans” category 526A. As shown in FIG. 2E, a doctor's assignment of a care plan to a patient can be categorized by processing module 60 accordingly under the “Care Plan” category 526A and displayed on a corresponding “Care Plan” page of user interface 510.

In some embodiments, patient engagement system 10 and its user application 501 are configured to allow an authenticated user to access the details of a sub-event 524A. For example, an authenticated user may select the sub-event 524A corresponding to the assignment of a care plan to view the details of the assigned care plan as illustrated in FIG. 2F. In the example illustrated in FIG. 2F, the details of a sub-event 524A categorized under the “Care Plan” category 526A include a field summarizing the patient outcomes, a field summarizing the recommended follow-up appointments, and a field summarizing the patient's prescription.

Other aspects of the invention relate to a patient engagement system 10 that may be adapted to share a patient's personal health information with authorized individuals (e.g. guardians, family members, other participates in patient engagement, etc.). Patient engagement system 10 may optionally comprise an information sharing module 70 configured to grant authorized individuals with a single point of access to a patient's health information. In some embodiments, information sharing module 70 is configured to obtain the identity and/or contact information of the authorized individuals directly from an authenticated user of application 501. That is, an authenticated patient can provide the identity and/or contact information of individuals that they wish to authorize (i.e. to grant access to their personal health information) to user application 501, and user application 501 will communicate the provided information to user application module 40 of patient engagement system 10.

FIG. 2G illustrates an example of a page 521 of user interface 510 for inviting and/or displaying a list of authorized individuals. As illustrated in FIG. 2G, the “Supporters” page 521 may contain instructions that prompt a patient to add new individuals to their list of authorized individuals (e.g. by providing the new individual's name, contact information, etc.). Like the “Results” and “Care Plans” pages, page 521 may include a list of sub-events 524A corresponding to activities performed by the authorized individuals (not shown). In some embodiments, menu 526 comprises a shortcut icon corresponding to page 521 and the shortcut icon may be selected by the user to quickly navigate to page 521.

In some embodiments, information sharing module 70 is configured to implement a tiered health information sharing system. That is, different authorized individuals may be categorized into different tiers with different permission levels of access to the patient's personal health information. For example, authorized individuals who are categorized into a higher tier may be granted access to the detailed medical test results and care plans of the patient, whereas authorized individuals who are categorized into a lower tier may be granted access to only summary information provided on the homepage 520 of user interface 510.

In some embodiments, the different tiers and/or permission levels of access are determined or otherwise set by the patient at the time of providing the authorized individual's contact information into user application 501. That is, an authorized individual's contact information and the tier and/or permission level of access to be granted thereto may be communicated together to user application module 40. This allows the patient to pre-determine and information that is accessible to the authorized individuals before the authorized individuals are granted access to patient engagement system 10.

In some embodiments, mobile application 501 is configured to allow a patient to adjust the tier and/or permission level of access of an authorized individual at any time. That is, a patient user of device 500 can navigate to page 524 and upgrade or downgrade an authorized individual's tier and/or permission level of access by interacting with the features contained therein. This empowers the patient to control the accessibility of their own personal health information.

Systems and methods according to embodiments as described herein may possess one or more of the following features or characteristics:
patient identification instruments 100 (e.g. a hospital armband or wristband) can, through patient engagement system 10, function as a two-way communication tool for providing information to patients, as opposed to a one-way communication tool for providing information to only the staff of a health facility;
patients can, through their own user devices 500, connect with and access digital resources already in use by various healthcare facilities around the world;
the system is compatible with patient identification instruments 100 that are already in use and/or patient identification instruments 100 that are printed by standard laser printers or direct thermal printers;
the system is user-friendly and easy to use for patients and/or individuals authorized to access their health information;
the system can be operated in real time to provide real time updates to the authenticated users;
the system can be independently operated by a third party service provider, so it does not introduce additional overhead costs to the existing healthcare facilities;
the system ensures patient privacy and complies with privacy regulations set by government regulators;
the system can be customized to a healthcare facility's needs (e.g. patient registration information 221 required to authenticate a user may vary among different health facilities, each health facility may have different digital resources that are made available to the patient);
they system empowers the patient by, for example, allowing the patient to provide feedback in real time, to improve patient engagement;
the system engages patients in their care journey from the beginning to the end.

The examples and corresponding diagrams used herein are for illustrative purposes only. Different configurations and terminology can be used without departing from the principles expressed herein.

Although the invention has been described with reference to certain specific embodiments, various modifications thereof will be apparent to those skilled in the art without departing from the scope of the invention. The scope of the claims should not be limited by the illustrative embodiments set forth in the examples, but should be given the broadest interpretation consistent with the description as a whole. For example, various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

1. A computer-implemented method of authenticating a mobile device of a patient for access to a patient engagement system, the method comprising:
receiving patient registration information from a health information system over a first network;
receiving patient authentication information from the mobile device of the patient over a second network, the patient authentication information obtained from a patient identification instrument issued to the patient by a provider of the health information system;
comparing the patient registration information with the patient authentication information; and
where the patient registration information matches the patient authentication information, generating a confirmation message comprising a verification code or confirmation link and sending the generated confirmation message to the mobile device using contact information;
receiving a communicated confirmation message from the mobile device, wherein the communicated confirmation message includes the verification code or is generated using the confirmation link;
where the verification code in the communicated confirmation message matches the verification code in the generated confirmation message or the communicated confirmation message was generated using the confirmation link, granting the mobile device with access to the patient engagement system.

2. The method of claim 1, wherein the patient authentication information is obtained by the mobile device reading a visual machine-readable code printed on the patient identification instrument.

3. The method of claim 2, wherein the patient authentication information is decoded from the visual machine-readable code at the mobile device before the patient authentication information is received at the patient engagement system.

4. The method of claim 2, wherein the patient authentication information is decoded from the visual machine-readable code at the patient engagement system.

5. The method of claim 1, wherein the provider of the health information system is a hospital and the patient identification instrument is a hospital wristband.

6. The method of claim 1, comprising:
where the patient registration information matches the patient authentication information, creating a patient profile of the patient based on the patient registration information, the patient profile including a contact information of the patient obtained from the patient registration information.

7. (canceled)

8. The method of claim 1, wherein the first network is a secure private network and the second network is a public network.

9. The method of claim 1, wherein the first network is a public network and the second network is the public network.

10. The method of claim 1, comprising receiving the patient registration information from the health information system over the first network in real-time.

11. The method of claim 10, comprising retrieving patient registration information from the health information system upon receipt of the patient authentication information.

12. A patient engagement system comprising:
an interface module for receiving patient information from a health information system, the patient information including patient registration information and patient medical information;
a user application module for receiving user information and a communicated confirmation message from a mobile device, the user information including patient authentication information, and the communicated confirmation message includes a verification code or is generated using a confirmation link;
a user authentication module for comparing the patient registration information with the patient authentication information and for, where the patient registration information matches the patient authentication information, generating a confirmation message comprising a the verification code or the confirmation link and sending the generated confirmation message to the mobile device using contact information;
a database for storing the patient medical information and the user profile; and a processing module for managing access to the patient medical information based on one or more access rules, the one or more access rules defined at least in part by the user profile.

13. The patient engagement system of claim 12, wherein the interface module is connected to the health information system by way of a secured application programming interface (API) provided by a provider of the health information system.

14. The patient engagement system of claim 12, wherein the user application module is connected to the mobile device by way of the internet.

15. The patient engagement system of claim 12, wherein the database is a cloud-based database residing behind one or more layers of security firewalls.

16. The patient engagement system of claim 12, wherein the health information system is a hospital information system.

17. (canceled)

18. (canceled)

19. The method of claim 1, wherein the mobile device is configured to automatically detect receipt of the generated confirmation message and automatically communicate the communicated confirmation message.

20. The method of claim 1, wherein the contact information comprises at least one of a telephone number and an email associated with the mobile device.

21. The method of claim 1, further comprising adjusting a level of authentication required based on at least one of a location of the mobile device, a time of access, and a type of medical information being accessed.

22. The patient engagement system of claim 12, wherein the mobile device is configured to automatically detect receipt of the generated confirmation message and automatically communicate the communicated confirmation message.

23. The patient engagement system of claim 12, wherein the contact information comprises at least one of a telephone number and an email associated with the mobile device.
